(19) United States
(12) Patent Application Publication
(10) Pub. No.: US 2012/0121636 A1
(43) Pub. Date: May 17, 2012

(54) Tat-Based Vaccine Compositions and Methods of Making and Using Same

(75) Inventor: David I. Cohen, Pelham, NY (US)

(73) Assignee: NANIRX, INC., New York, NY (US)

(21) Appl. No.: 13/354,986

(22) Filed: Jan. 20, 2012

Related U.S. Application Data
(63) Continuation of application No. 12/405,086, filed on Mar. 16, 2009, now abandoned.
Application No. 12/405,086 is a continuation of application No. 10/598,975, filed on Sep. 15, 2006, now abandoned.
Application No. 10/598,975 is a continuation of application No. 10/456,865, filed on Jun. 6, 2003, now abandoned.
Application No. 10/456,865 is a division of application No. 09/636,057, filed on Aug. 10, 2000, now Pat. No. 6,667,151.
(60) Provisional application No. 60/553,733, filed on Mar. 16, 2004.

(51) Int. Cl. A61K 39/39; A61P 31/20; A61P 31/00; A61P 35/00; A61K 35/76; A61P 37/04

(52) U.S. Cl. 424/188.1; 424/192.1; 424/281.1

(57) ABSTRACT

A Tat-based vaccine composition comprising at least one antigen coupled to at least one immunostimulatory lentivirus trans-activator of transcription (Tat) molecule wherein the antigen is a cancer antigen an infectious disease antigen or a fragment thereof and methods to treat disease by administering the Tat-based vaccine composition. An additional Tat-based vaccine composition comprising immunostimulatory lentivirus Tat is provided.

### RELATED APPLICATIONS

The present application is a continuation of U.S. patent application Ser. No. 12/405,086, filed Mar. 16, 2009, which is a continuation of U.S. patent application Ser. No. 10/598,975, filed Sep. 15, 2006, which is a National Phase Application under 371 of PCT/US2005/008519 filed on Mar. 16, 2005, which claims priority to U.S. Provisional Patent Application Ser. No. 60/553,733 filed Mar. 16, 2004 and to U.S. patent application Ser. No. 10/456,865 filed Jun. 6, 2003, now abandoned, which is a divisional of U.S. patent application Ser. No. 09/636,057 filed Aug. 8, 2000, now U.S. Pat. No. 6,667,151. The disclosures of all of these patent applications are incorporated by referenced herein in their entirety.

### FIELD OF THE INVENTION

The present invention relates to the field of immune modulation therapeutics and more specifically to vaccine compositions useful for induction of stimulatory immune responses for the prophylaxis and treatment of cancer and infectious. Specifically, the vaccine compositions of the present invention are based on immunostimulatory variants of human immunodeficiency virus trans-activator of transcription (Tat), or fragments thereof, conjugated to an antigen. Vaccine compositions are also provided which comprise immunostimulatory Tat and optionally antigen. Additionally, methods of treating cancer and infectious diseases using the Tat-based vaccine compositions of the present invention are provided.

### BACKGROUND OF THE INVENTION

Recently, significant advances have been made in understanding the human immunodeficiency disease (HIV) process. For many years, researchers have been unable to explain the seemingly immediate and profound destruction of the immune system following the initial HIV infection. Equally puzzling was a phenomenon seen in a few patients referred to as long term non-progessors (LTNP). In LTNP patients, viral loads are high and the virus can be isolated easily from the HIV target immune cells such as CD4+ T lymphocytes (referred to herein as T4 cells). However, unlike the majority of infected individuals who develop acquired immune deficiency syndrome (AIDS), the LTNP do not demonstrate significant reduction in their T4 cells and do not progress to AIDS.

One possible, non-binding, theory that may explain these two phenomena involves a non-structural protein (a protein encoded by the virus genome that is not actually part of the virus itself) called the trans-activator of transcription (Tat). Tat is a variable RNA binding peptide of 86 to 110 amino acids in length that is encoded on two separate exons of the HIV genome. Tat is highly conserved among all human lentiviruses and is essential for viral replication. When lentivirus Tat binds to the TAR (trans-activation responsive) RNA region, transcription (conversion of viral RNA to DNA then to messenger RNA) levels increase significantly. The Tat protein associated with lentivirus virulence will be referred to hereinafter as Tat, Recently, it has been demonstrated that Tat increases viral RNA transcription and it has been proposed that Tat may initiate apoptosis (programmed cell death) in T4 cells and macrophages (a key part of the body's immune surveillance system for HIV infection) and possibly stimulates the over production of alpha interferon (α-interferon is a well established immunosuppressive cytokine). These, and other properties of lentivirus Tat proteins, have led to considerable scientific interest in Tat's role in pathogenesis and to the present inventors proposal that Tat may act as a powerful immunosuppressant in vivo.

A potential key to lentivirus Tat pathogenesis may involve in its ability to trigger apoptosis. Conventional Tat initiates apoptosis by stimulating the expression of Fas ligand (FasL, a monomeric polypeptide cell surface marker associated with apoptosis) on the T4 cell and macrophage surface. When FasL is cross linked by binding with Fas (the counter part to FasL which is also expressed on a wide variety of cell types), the apoptotic system is activated. Consequently, the death of these essential T4 cells and macrophages is accelerated, resulting in extreme immunosuppression. Thus, extracellular Tat's presence early in the course of HIV infection could reduce a patient's immune response, giving the virus an advantage over the host. Furthermore, the direct destruction of T4 cells and induction of α-interferon production could help explain the lack of a robust cellular immune response seen in AIDS patients, as well as accounting for the initial profound immunosuppression.

Further support for this concept is found in a surprising new observation made by the present inventor who has demonstrated the Tat protein isolated from long term non-progressors is different from C-Tat found in AIDS patents. The Tat protein found in LTNP is capable of trans-activating viral RNA, however, LTNP Tat (designated herein after as IS-Tat for immunostimulatory Tat) does not induce apoptosis in T4 cells or macrophages and is not immunosuppressive. Moreover, T4 cells infected ex vivo with HIV isolated from LTNP (such cell lines are designated Tat TcL) can result in the over expression of IS-Tat proteins, often to the virtual exclusion of other viral proteins, that are strongly growth promoting rather than pro-apoptotic. The tat genes cloned from these Tat TcLs reveal sequence variations in two tat regions, at the amino terminus and within the first part of the second exon. These surprising discoveries could help explain why HIV infected LTNP T4 cells do not die off at the staggering rate seen in HIV infected individuals that progress to AIDS.

Additionally, variants of Tat are found in lentiviruses which infect monkey species yet do not result in the development immunodeficiency and epidemic infection. These variant Tat proteins direct monocyte differentiation into DCs which stimulate CTL responses. These simian Tat variants, and other Tat variants that are not immunosuppressive, have been termed attenuated or immunostimulatory Tat (IS-Tat).

Based on the observations with long-term CD4+ Tat T cell lines (Tat TcL), clinical observations, and experiments in animals, attenuated Tat (more specifically IS-Tat or, alternatively, Tat proteins that have been chemically or physically altered) may act as an immune stimulant activating T4 cells inducing their proliferation. This principle may help to explain the stable T4 levels seen in LTNP. Moreover, attenuated Tat may be useful as an adjuvant when co-administered with other active vaccine components such as, but not limited to, vaccines for other viruses, bacteria, rickettsia and cancer cells.

Cancers and chronic infections are the most prominent examples of common human diseases that respond to immune-based treatments. Although infections were the first diseases to be controlled by immunization, a series of clinical trials in humans starting in the 1980s have established that an immune response, particularly of the cytotoxic T lymphocyte (CTL) arm of the immune system, could regress some human melanomas (Phan C Q, et al., Cancer regression and autoimmunity induced by cytotoxic T lymphocyte-associated antigen 4 blockade in patients with metastatic melanoma, Proc Natl Acad Sc. USA 100:8372-7, 2003) and renal cancers. These observations were broadened by the discovery that dendritic cells (DC), a specific class of antigen-presenting cells (APC), are particularly effective at initiating CTL activity against cancers and other diseases (Banchereau J et al., Dendritic cells as vectors for therapy, Cell 106:271-4, 2001; Dalyot-Herman N et al., Reversal of CD8+ T cell ignorance and induction of anti-tumor immunity by peptide-pulsed APC, J Immunol 165:6731-7, 2000). Technologies that target and activate DC have yielded some early successes against human cervical pre-malignancies, caused by infection with Human Papilloma Virus (HPV) and human lung cancer. In contrast to chemotherapeutic drugs currently used against cancer, agents that provoke a CTL response against cancer potentially are accompanied by few side effects, owing to the great specificity of the immune response.

Efforts to develop immunotherapeutic drugs that treat cancer have been hampered by technical difficulties in targeting and activating DC to deliver and sustain the required entry signals to the CTL. Antigen targeting for the induction of a CTL response is a challenge insofar as natural processing requires that the antigen enter the cytoplasm of the cell in order to bind to the immune system's major histocompatibility complex (MHC) Class I antigen, a prerequisite to CTL activation because the ligand for activating the T cell receptor on CTL is a complex of antigen and MHC Class I. In almost all cases protein antigens, even when they are coupled with a DC co-activator, enter exclusively into the alternative MHC Class II antigen presentation pathway that excludes CTL stimulation. This can be overcome in part by peptide-based technologies, because peptides bind to MHC Class I that is already on the surface of the DC. However, this technology is non-specific and most peptides are poor DC activators which limits their efficacy as human treatments for cancer.

A limited group of biological proteins are known to stimulate a CTL response. Variants and derivatives of the Human Immunodeficiency Virus 1 (HIV-1) trans-activator of transcription (Tat) can stimulate this CTL response (Moy P et al., Tat-mediated protein delivery can facilitate MHC class I presentation of antigens, Mol Biotechnol 6:105-13, 1996; Fanales-Belasio E et al., Native HIV-1 Tat protein targets monocyte-derived dendritic cells and enhances their maturation, function, and antigen-specific T cell responses, J Immunol 168:197-206, 2002). Additional biologics that are currently known to directly trigger a CTL response are based on heat shock proteins (HSP) (Suzue K et al., Heat shock fusion proteins as vehicles for antigen delivery into the major histocompatibility complex class I presentation pathway, Immunol 94:13146-51, 1997; Stebbing J et al., Disease-associated dendritic cells respond to disease-specific antigens through the common heal shock protein receptor, Blood 102:1808-14, 2003), or on the outer coat protein of certain bacteria. Heat shock proteins have shown limited efficacy in the treatment of certain genital neoplasms related to HPV infection.

A large body of evidence implies that Tat is secreted from infected cells. Extracellular Tat is taken up by uninfected cells resulting in trans-activation of transcripts, a subset of which stimulate the cell (Frankel A D and Pabo C O, Cellular uptake of the Tat protein from Human Immunodeficiency Virus, Cell 55:1189-93, 1988) and a subset of which initiate programmed cell death. These observations demonstrate that Tat enters the cytoplasm of cells, where trans-activation is mediated, but they did not establish the key mechanism of entry via the receptor. The immediate immunosuppression that accompanies HIV infection has been attributed to Tat and has hindered the generation of successful HIV vaccines (Viscidi R P et al, Inhibition of antigen-induced lymphocyte proliferation by Tat protein from HIV-1, Science 146:1606-8, 1989; Cohen S S et al., Pronounced acute immunosuppression in vivo mediated by HIV-1 Tat challenge, Proc Natl Acad Sci USA 96:10842-47, 1999). Additionally, Tat suppression occurs at both the antibody level and at the T cell level and is antigen-specific. This distinguishes Tat-induced immunosuppression from other immunosuppressants currently used in human therapy, such as cyclosporine, that work exclusively on T cells.

Biological agents currently used to treat disease introduce foreign antigens (monoclonal antibodies, insulin, Factor VIII, organ transplants) into the body. An immune response against these antigens is undesirable because this immunity neutralizes, or in the case of organ transplants, rejects the foreign body in addition to causing collateral damage through allergic and autoimmune reactions. Recombinant proteins of human origin have been very successful in overcoming this problem and sustaining the efficacy of certain biological therapies such as insulin, Factor VIII, and monoclonal antibodies. However, even in these successes, undesired auto-antibodies can still accumulate over time that limit or terminate efficacy. Methods to ameliorate these undesirable immune responses have not yet been developed.

Current immunosuppression treatment regimens are primarily designed for organ transplantation where a highly immunogenic foreign body often with multiple foreign antigens (histocompatibility antigens) must be maintained for the life of the patient. Up till the present time, this involves non-specific suppression of the entire immune system with multiple agents. Physicians and researchers have devised therapeutic regimens where a balance between the side effects of the immunosuppressants and organ rejection can be reached. The most common side effects associated with common immunosuppressive cocktails, which can include corticosteroids, cyclosporine and azathioprine, include stunted growth, weight gain, bone marrow inhibition, anemia, low white blood cell count and kidney damage. The most serious side effects, however, are infection, particularly with viruses and tumor formation due to the non-specific nature of the immune suppression. Therefore there exists a need to improved antigen-specific immunosuppressive therapies.

Autoimmune diseases are a series of unwanted immune responses that selectively destroy tissues. Severe autoimmune diseases are chronic, debilitating, and life-threatening. In some cases, specific agents that provoke a particular type of autoimmune disease are becoming defined. Approximately 2.5 million individuals currently suffer from rheumatoid arthritis (RA) in the US alone. Severe RA accelerates death rates at least five-fold compared to the general population (Wolfe F et al., Predicting mortality in patients with RA, Arth Rheumatism 48:1530-42, 2003). Peptide fragments from collagen type II, an important structural component in undamaged joints, can provoke RA in animals and could be developed as tolerizing agents for use against human RA (Van den Steen P et al., Cleavage of denatured natural collagen type II by neutrophil gelatinase B reveals enzyme specificity, post-translational modifications in the substrate, and the formation of remnant epitopes in rheumatoid arthritis, FASEB J 16:379-89, 2002).

Therefore, there exists a medical need for compositions which can be used as vaccines to specifically stimulate desired immune responses, such as in infectious diseases or cancer, and other compositions that suppress inappropriate immune responses to certain therapeutic, diagnostic or prophylactic agents and in autoimmune diseases in an antigen-specific manner.

### SUMMARY OF THE INVENTION

For the purposes of clarification and to avoid any possible confusion, the HIV Tat as used in the vaccine compositions of the present invention will be designated as either “Tat” for conventional immunosuppressive Tat protein and “Tat*” or “ox-Tat*” for Tat that is genetically or chemically derivatized so that it is stimulatory. Additional abbreviations for Tat used in this disclosure include sTat (soluble Tat) and C-Tat (conventional native immunosuppressive Tat from HIV).

The present invention provides vaccine compositions for induction of stimulatory immune responses for the prophylaxis and treatment of infectious diseases and cancer. The vaccine compositions of the present invention are based upon immunostimulatory variants of the human immunodeficiency virus (HIV) trans-activator of transcription (Tat). The vaccine compositions of the present invention are constructed from Tat, or Tat fragments, that have been derivatized to be stimulatory and conjugated to antigens, or antigen fragments. The vaccine compositions of the present invention can be constructed though a variety of means known to persons skilled in the art including, but not limited to, protein conjugation, avidin-biotin conjugation, genetically engineered molecules and the like.

In an embodiment of the present invention, a Tat-based vaccine composition is provided comprising at least one antigen coupled to at least one immunostimulatory lentivirus trans-activator of transcription (Tat) molecule. The antigen can be a cancer antigen or an infectious disease antigen, or a fragment thereof. Non-limiting examples of cancer antigens useful in the vaccine compositions of the present invention include antigens associated with cell growth and human papilloma virus E7 antigen.

In an embodiment of the present invention, the immunostimulatory lentivirus Tat is oxidized human immunodeficiency virus-1 (HIV-1) Tat. In another embodiment of the present invention, the immunostimulatory lentivirus Tat is the human HIV-1 Tat wherein the amino acid proline at positions 6, 10 and 14 of SEQ ID NO. 1 is replaced with the amino acid glycine. In yet another embodiment of the present invention, the immunostimulatory lentivirus Tat comprises the amino acid sequence of SEQ ID NO. 11.

In another embodiment of the present invention, the immunostimulatory lentivirus Tat and antigen of the Tat-based vaccine composition are linked through genetic engineering of their DNA to provide a recombinant protein.

In an embodiment of the present invention, a method is provided for treating cancer or infectious diseases comprising administering at least one Tat-based vaccine composition to a patient in need thereof.

In another embodiment of the present invention, a Tat-based vaccine composition is provided comprising immunostimulatory lentivirus Tat and optionally at least one antigen wherein the antigen is a cancer or infectious disease antigen, or a fragment thereof. In an embodiment of the present invention, the immunostimulatory lentivirus Tat is oxidized human immunodeficiency virus-1 (HIV-1) Tat. In another embodiment of the present invention, the immunostimulatory lentivirus Tat is the human HIV-1 Tat wherein the amino acid proline at positions 6, 10 and 14 of SEQ ID NO. 1 is replaced with the amino acid glycine. In yet another embodiment of the present invention, the immunostimulatory lentivirus Tat comprises the amino acid sequence of SEQ ID NO. 11.

### BRIEF DESCRIPTION OF THE DRAWINGS

FIG. 1 depicts fluorescence activated cell sorter analysis of the results of Tat activation of monocytes according to the teachings of the present invention. Human peripheral blood monocytes were committed to differentiate into DCs through 5 days of culture in GM-CSF and IL-4. Committed DCs were cultured overnight either in medium alone (Control), LPS, or Tat, after which they were stained with an anti-CD86 antibody and analyzed by FACScan for CD86, a specific marker of DC activation, induction (left panel) or generalized activation (right panel, enlargement into box R2, shown for Tat-stimulated cells).

FIG. 2 depicts the enhancement of antigen-specific activation of CTLs by Tar-antigen (Ag) complexes according to the teachings of the present invention. CTL activity was quantitated as the number of γ-interferon-secreting spot-forming colonies (SFC)/10⁶ plated cells using ELISPOT assays.

FIG. 3 depicts median fluorescence of monocytes, cultured for six days either with no stimulus (0), TNF-α, LPS, decreasing concentrations of C-Tat, or oxidized ox-C-Tat and stained with an anti-Fas ligand (FasL) monoclonal antibody (Mab) followed by a fluorescinated goat anti-mouse polyclonal antibody.

FIG. 4A-B depicts antibody titer to immunizing antigen administered with the tolerogen composition of the present invention (PT) or non-immunosuppressive ox-Tat* (Ag) at 2 weeks (A) and 6 weeks (B) after immunization.

FIG. 5 depicts fluorescence-activated cell sorter analysis of mouse peritoneal macrophages that were isolated either after in vivo thioglycolate stimulation (Stimulated+adjuvant) or without in vivo stimulation (resting). Mouse peritoneal macrophages were cultured for five days either in the absence of additional stimulation (C), with LPS or with Tat. Activation was determined as percent enlarged cells (M1 fraction).

FIG. 6 depicts stable suppression of antigen-stimulated T lymphocytes by Tat-Ag complexes two weeks after immunization with the tolerogen compositions of the present invention.

FIG. 7 depicts the antigen-specificity of Tat suppression according to the teachings of the present invention. Mice were immunized at day 0 and boosted at day 7 with an adjuvant emulsion containing either Tat (Ag+Tat), or with Ag Alone as control. At day 14, draining lymph node cells were harvested and stimulated with either specific or non-specific antigen and proliferation measured by ³H thymidine uptake (CPM) after four days of culture.

FIG. 8 depicts fluorescence-activated cell sorter analysis of human peripheral blood monocytes cultured for four days in control medium (Control), or medium containing Tat or LPS according to the teachings of the present invention. Harvested cells were doubly stained with a fluoresceinated anti-FasL Mab (αFasL-fitc) and with an anti-CD14 rhodamine labeled Mab. Cells were analyzed by FACScan for activation (forward scatter), CD14 expression (% macrophages, R2), and for induction of Fas ligand (MFI). The T cell population is labeled R1.

FIG. 9A-B depicts the regulatory and immunosuppressive characteristics of Tat-activated macrophages according to the teachings of the present invention. (A) Human polymorphonuclear neutrophils (PBMC) from one individual (PBMCs #3) cultured for 5 days in either medium with tetanus antigen (Ag), antigen with the further addition of Tat (Ag+Tat) or Ag with Tat and recombinant sFas protein (Ag+Tat+sFas). The results are graphed as stimulation index (mean cpm stimulated culture/mean cpm medium control). (B) Proliferation of PBMCs cultured 6 days with either tetanus or Candida antigen alone (Ag), compared with cultures in which Tat (Ag+Tat), or Tat and the antagonistic anti-Fas antibody, ZB4, were added (Ag+Tat+αFas).

FIG. 10 depicts domain 1 of the Tat molecule, the signal transduction domain, amino acids 3-19.

FIG. 11 depicts domain 2 of the Tat molecule, the cysteine-rich ligand binding domain, amino acids 22-37.

FIG. 12 depicts domain 3 of the Tat molecule, the membrane translocation sequence, amino acids 47-57.

FIG. 13 schematically depicts the construction of vaccine and tolerogen cassettes according to the teachings of the present invention. Panel A: Domains of native Tat. Panel B: Varying antigen cassettes for the production of the vaccines or tolerogens of the present invention. The immunostimulatory or immunosuppressive functions of domain 1 (SH3 binding motif) will determine if the resultant protein is a vaccine (immunostimulant) or tolerogen (immunosuppressive).

FIG. 14. depicts tolerogen composition constructs according to the present invention specific for preventing immune responses to human or humanized monoclonal antibodies.

FIG. 15. depicts re-activation of T lymphocytes by cytokines and vaccine compositions made according to the teachings of the present invention.

FIG. 16 depicts the efficacy of cancer vaccine compositions made according to the teachings of the present invention in shrinking tumor size and improving survival in a mouse model of cervical cancer.

### DETAILED DESCRIPTION OF THE INVENTION

The present invention provides Tat-based vaccine compositions that activate dendritic cells (DC) and antigen-specific cytotoxic T lymphocytes (CTL) for the prophylaxis and therapy of cancer and infectious diseases. The present invention describes derivatizations in the Tat molecule that could improve its ability to activate DC and methods to design novel adjuvants that could substitute for the DC-activating effects of derivatized Tat.

For the purposes of clarification and to avoid any possible confusion, the HIV Tat as used in the vaccine compositions of the present invention will be designated as either “Tat” for conventional immunosuppressive Tat protein and “Tat*” or “ox-Tar” for Tat that is genetically or chemically derivatized so that it is stimulatory. Additional abbreviations for Tat used in this disclosure include sTat (soluble Tat) and C-Tat (conventional native immunosuppressive Tat from HIV).

The vaccine compositions of the present invention are constructed from derivatized Tat, or Tat fragments, conjugated to cancer or infectious disease antigens, or antigen fragments. The vaccine compositions of the present invention can be constructed though a variety of means known to persons skilled in the art including, but not limited to, protein conjugation, specific cross-linking methods, creation of recombinant molecules and the like.

The present inventor has unexpectedly demonstrated that HIV-1 Tat mediates two independent activities, a receptor-mediated triggering event at the cellular surface and an intracellular trans-activation activity that controls antigen-presenting cell (APC) differentiation. The receptor-mediated triggering event mediated by Tat is specific to APC, committing them for activation and differentiation into highly immunosuppressive antigen presenting cell regulatory macrophages (AReg) or into dendritic cells (DC) that stimulate specific cytotoxic T lymphocytes (CTL).

Antigen presenting cells, macrophages and dendritic cells are critical in the pathogenesis of responses to a variety of diseases, disorders and undesirable or inappropriate immune responses.

The vaccine compositions of the present invention can be stably produced as recombinant molecules or as direct protein conjugates. In one embodiment of the present invention, the DNA sequence of an antigen, to which an immune response is desired, is inserted into a vaccine expression cassette and the Tar-antigen construct is produced by growing the vaccine expression cassette in the appropriate cell system. Any antigen to which an immune response is desired is suitable for incorporation into the vaccine composition of the present invention. Suitable cancer antigens include, but are not limited to, human papilloma virus (HPV) E6 and E7 in cervical carcinoma, the MAGE series of antigens MAGE-1, MAGE-2, MAGE-3, MART-1/melanA, gp100, MC1R, tyrosinase, the gangliosides GD2, O-acetylated GD-3 and GM-2, urinary tumor-associated antigens, breast cancer antigens including lactalbumin and its derivatives, glycosylated surface molecules including the antigen recognized by the TAG 72 monoclonal antibody, and E cadherin, and over 50 antigens that have been detected in pancreas cancer.

In one embodiment of the present invention, derivatized Tat* protein, or a fragment thereof, is chemically coupled to a desired antigen to produce a vaccine composition. In a non-limiting example, these conjugates are simply linked using a widely known biotin-avidin system. Biotin, a vitamin, and avidin, a lectin, have a high affinity to one another such that proteins conjugated to biotin bind in a stable manner to proteins conjugated to avidin. Derivatized Tat* is biotinylated using methods well known to a person of ordinary skill in the art. Similarly, the antigen of interest is conjugated to avidin according to standardized methodology. When biotinylated Tat* and avidin-Ag are combined under concentration and temperature conditions necessary for such a reaction, a Tat*-Ag conjugate is formed. It is within the scope of the present invention to conjugate antigens and derivatized Tat* by other methods known to those skilled in the art of protein chemistry.

In order to make the Tat-based vaccine compositions of the present invention, it is necessary to remove, modify, or override through mutation, the suppressive elements in Tat such that DC activation is maintained. Based upon structural resolutions, the present inventor describes a critical SH3 binding domain within the Tat sequence that controls the generation of a highly immunosuppressive antigen presenting cell regulatory macrophages (AReg). Simian lentiviruses related to HIV-1, but not causing immunodeficiency, have an alternative domain that is not suppressive. A mutant protein in a mouse strain (hairless, hr) that develops an immunodeficiency strikingly parallel to that seen in HIV-1 infection, including lost CTL and poor APC functions, encodes a SH3 binding domain homologous to HIV-1 Tat. This SH3 domain is proposed to control the differentiation potential of monocyte precursors either into DC that stimulate CTL, or AReg that suppress CTL. While prominent in HIV-1 infection, AReg are also now recognized as critical contributors to invasion of gastric (Ishigami S et al., Tumor-associated macrophage (TAM) infiltration in gastric cancer, Anticancer Res 23:4079-83, 2003), pancreas (von Bernstorff W et al., Systemic and local immunosuppression in pancreatic cancer patients, Clin Cancer Res 7:925s-32s, 2001), and ductal infiltrating breast tumors (Lin E Y et al., The macrophage growth factor CSF-1 in mammary gland development and tumor progression, J. Mammary Gland Biol Neoplasia 7:147-62, 2002; Visscher D W et al., Clinicopathologic analysis of macrophage infiltrates in breast carcinoma, Pathol Res Pract 191:1133-9, 1995), as well as components of tolerance in organ transplantation.

Recent surprising discoveries by the present inventor demonstrate that Tat can trigger CTL responses when its DC stimulatory activity is isolated away from the suppression derived from AReg activation. It has not been previously possible to extend the in vitro activities of Tat to animals. One obstacle was a failure to understand that the cellular target of Tat activity was a precursor APC as opposed to the T cell, as had been widely believed. The present inventor determined that Tat stimulates APCs, as opposed to T cells and other cell types, at picomolar concentrations that are physiologic for in vivo activity. In vitro, APCs are approximately 1000 times more sensitive to Tat than T4 lymphocytes. Due to this discovery, one barrier to the successful use of Tat as an immunotherapeutic, namely achieving concentrations attainable in vivo, has been overcome. Thus, Tat has two activities that are the core of the present invention; APC targeting and induction of antigen-specific effects that result from APC activation.

Therefore, an embodiment of the present invention illustrated in Example 2 is that derivatized Tat* induces monocytes committed to the DC lineage to enlarge into activated, CD86+ DC APCs. The effect of derivatized Tat* on this population of cells is stimulatory, rather than suppressive, because the cells have been previously committed to become DCs. The present inventor has previously demonstrated that chemically derivatized Tat (Tat* or ox-Tat) is immunostimulatory in that it promotes differentiation of monocytes into dendritic cells, which subsequently leads to antigen-specific activation of cytotoxic T lymphocytes. Therefore properly derivatized Tat* resulting from chemical or genetic modifications does not induce ARegs from monocyte APC precursors. Tat from HIV-1 long-term non-progressors (patients infected with HIV-1 who do not progress to Acquired Immune Deficiency Syndrome (AIDS)) and from certain related simian strains of lentivirus are also immunostimulatory rather than immunosuppressive. These natural variations in Tat are important sources of sequence modifications for the genetically-derivatized Tat* of the present invention.

The present invention presents a model of Tat activation of dendritic cells leading to activation of tumor-specific cytotoxic T lymphocytes. Owing to its monocyte targeting specificity, Tat enters APC precursors, carrying along with it any other protein conjugated to it. At this step the APC is stimulated. Once inside the APC, Tat can leave the endosome, the reservoir for almost all soluble proteins, and enter the cytoplasmic space, as indicated through its transactivation of RNA expression. This trafficking property of Tat causes the initiation of major histocompatibility complex (MHC) class I presentation, since association with MHC class I also only occurs in the cytoplasm. The balance and duration of cellular gene activation determines whether the APC differentiates into an activated DC that potently presents for CTL activation, or into an AReg that shuts off CTL and other immune responses. In Example 1, the Tat*-Ag vaccine composition of the present invention is genetically derivatized to favor sustained DC activation and thereby to stimulate a superior CTL response against a cancer, in one embodiment of the present invention, the antigen of interest is the E7 antigen associated with cervical cancer and human papilloma virus infection.

Tat contains three distinct regions of interest (Kuppuswamy M et al., Multiple function domains of Tat, the trans-activator of HIV-1, defined by mutational analysis, Nucleic Acids Res 17:3551-61, 1989). The first region of interest is the transduction domain at the amino terminus of Tat (amino acids 3-19). A second region of interest is a cysteine-rich proposed ligand binding domain (amino acids 22-37, SEQ ID NO. 7) which contains seven conserved cysteines. A third region of interest is the membrane translocation sequence (MTS) which encompasses amino acids 47-57. The complete amino acid sequence of HIV-1 Tat encoded by exons 1 and 2 of the Tat gene is depicted in SEQ ID NO. 1.

A proline rich stretch near the amino terminus (amino acids 3-19) of HIV-1 and HIV-2 Tat (SEQ ID NO. 3) within the transduction domain, has been described as a new SH3 binding domain having significant homology to the SH3-binding domain of the mouse hairless gene (hr) (SEQ ID NO. 4). Unexpectedly, mice expressing the hr gene mutation develop an AIDS-like syndrome characterized by poor CTL function, a shift in helper T lymphocytes from those regulating cell-mediated immunity (TH1) to those regulating antibody-mediated immunity (TH2) and increased susceptibility to chemical and ultraviolet light-induced skin cancers. Additionally, variants of Tat are found in lentiviruses that infect monkey species that do not develop immunodeficiency and that do not have epidemic infection. However, these variant Tat do not have the SH3 binding domain and instead substitute a different sequence, also set off by prolines at either end of the sequence, into the transduction domain. Therefore, this SH3 binding domain is central to the immunosuppressive activity of Tat. Genetic data indicates this SH3 binding domain regulates monocyte differentiation into ARegs. In Tat proteins which do not contain this SH3 domain or the domain is mutated, monocyte differentiation is directed into DCs which stimulate CTL responses.

It is also known that Tat contains a membrane translocation domain (MTS). After gaining access to the endosome following receptor binding, the MTS permits Tat to freely traffic across the endosomal membrane into the cytoplasm, where it transactivates gene expression, including but not restricted to genes of HIV-1 (Schwarze S R et al., In vivo protein transduction: delivery of a biologically active protein into the mouse, Science 285:1569-72, 1999). The MTS has been wrongly assumed to facilitate Tat entrance into the cell, which it can only accomplish at high concentrations that have been impossible to attain in vivo.

In an embodiment of the present invention, genetic derivatives of Tat, generated through modulating the signal transduction motif defined by the SH3 binding domain, are predicted to drive differentiation predominantly to dendritic cells or immunosuppressive AReg. AReg are also critical contributors to invasion of gastric, pancreas, and ductal infiltrating breast tumors, as well as components of tolerance in organ transplantation. It is a non-binding hypothesis of the present inventor that it is necessary to maintain the two external prolines at positions 3 and 18, flanking the SH3 domain in order to facilitate the proper structure for SH3 binding. In addition, the transduction domain from a non-immunosuppressive human variant Tat, or the domain from the hr mutation, can replace amino acids 3-19 of Tat (SEQ ID NO. 11), although the hr sequence (SEQ ID NO. 4) is predicted to increase suppression. In addition, the stimulatory simian form of Tat (SEQ ID NO. 5), or its human equivalent sequence (SEQ ID NO. 6), can be substituted at this domain. Additional chemical modifications, such as ox-Tat, can be used for stimulation of dendritic/CTL responses and synthetic chemical moieties (NICE, new immunomodulatory chemical entities) can be constructed to generate an equivalent response.

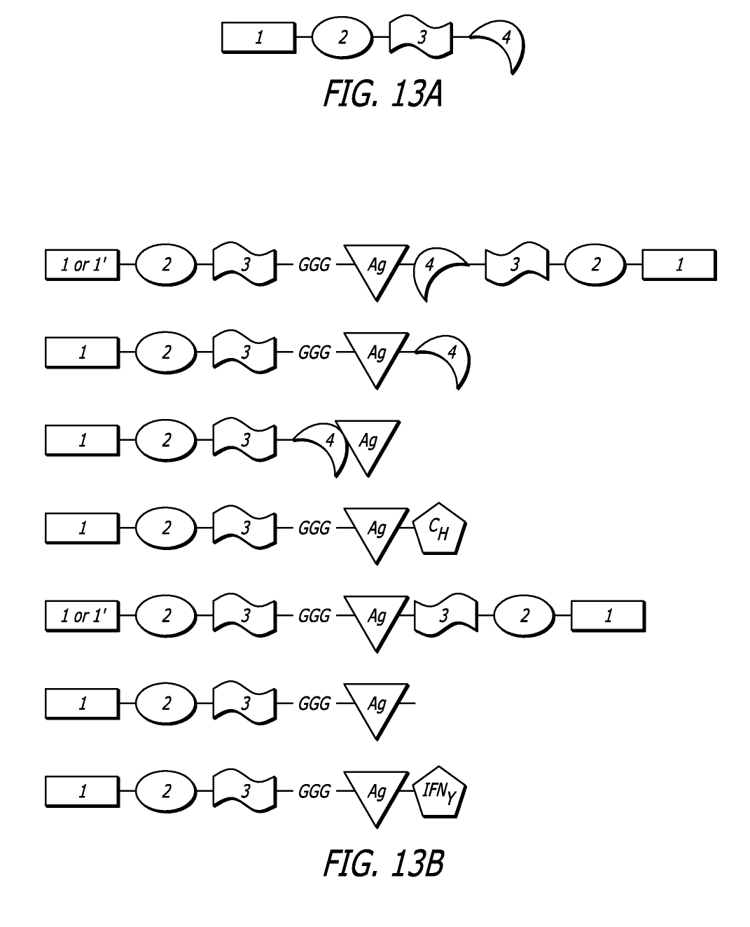

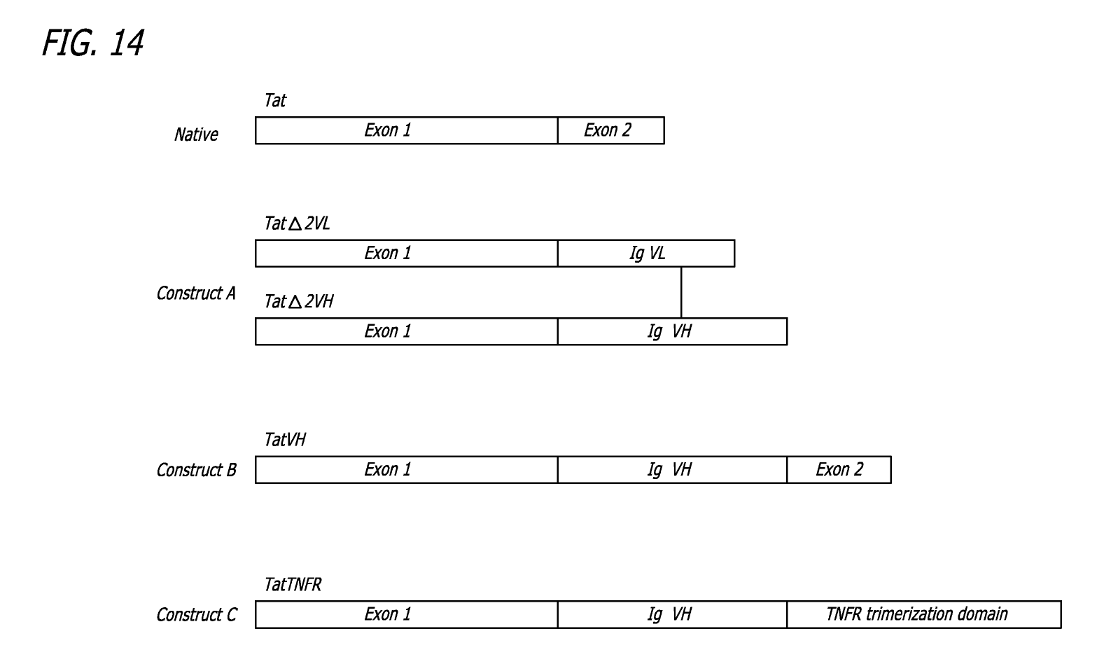

Variations and derivitizations of Tat for the purpose of stimulating an immune response in a vaccine composition are proposed in which Tat is conjugated to antigen in one of several proposed configurations and further illustrated in FIG. 13. The nature of the design allows the insertion of any specific antigen into a vaccine cassette described here, in which a beneficial immune response will result to that antigen. FIG. 13A represents native immunosuppressive HIV Tat with four domains: (1) the transduction (SH3) domain (amino acids 3-19); (2) the cysteine-rich ligand binding domain (amino acids 22-37, SEQ ID NO. 7); (3) the membrane translocation sequence (amino acids 47-57) and (4) a tail portion encoded by the second exon (amino acids 73-101). In all potential conformations presented in FIG. 13B, domain 1 can be the native immunosuppressive Tat SH3 domain (1) or a modified or mutated immunostimulatory SH3 domain (1′). The conformational structures of FIG. 13B represent potential conformation by which recombinant compositions can be constructed to provide the desired functional activity. Other conformations are anticipated to be within the scope of the present invention. The target antigen (Ag) is included in both tolerogen and vaccine compositions. Other potential components of the compositions of the present invention include immunoglobulin chains, or fragments thereof (CH) or other effector molecules such as interferon-γ (IFNγ).

The nucleotide sequences representing the components of the constructs in FIG. 13 are constructed in expression vectors and expressed in cellular expression systems known to persons skilled in the art. One exemplary expression system is the baculovirus expression system including the transfer plasmids pPSC12 and pPSC10 and BaculoKIT™ expression system from Protein Sciences Corp. (Catalog #1002, Meriden, Conn.). It is anticipated that other expression systems, include eukaryotic and prokaryotic systems, are within the scope of the present invention.

An additional therapeutic method to influence the SH3 control of dendritic cells involves activating RNA interference (RNAi), which results in sequence-specific degradation of the targeted double strand RNA (Fire A, RNA-triggered gene splicing, Trends Genet 15:358-63, 1999; Zamore P D, RNA interference: listening to the sound of silence, Nat Struct Biol 8:746-50, 2001). Small interfering RNAs (siRNA) are RNA duplexes of 21-23 nucleotides which activate the RNAi pathway through their antisense strand and silence a gene through targeted degradation of its transcript. siRNAs are being widely developed as prophylactic and therapeutic agents to suppress selected RNA transcripts. Proposed targets include oncoproteins in cancer and infectious agents. The specificity and sensitivity of the target, an opening on the transcript free from secondary structure or complexed proteins that allows duplexed siRNA to form, and the actual delivery of the siRNA drug inside the cell are three critical factors governing the outcome of treatment. The sequence of the SH3 binding domain predisposing AReg/DC outcome is a potential RNAi target. Because the Tat's activity occurs at a balance point between stimulation (DC) and suppression (ARegs), small perturbations can be extremely efficacious.

An embodiment of the current invention is to create vaccine compositions for cancer and infectious disease therapy using the genetic sequences discovered from analysis of Tat to control DC vs. AReg outcome. Duplexed siRNAs are constructed from the sense strand of Tat and Tat* variants using methods standard to those skilled in the art (Elbashir S M et al., RNA Interference is mediated by 21- and 22-nucleotide RNAs. Genes Devel 15:188-200, 2001). One of the obstacles associated with the successful therapeutic use of siRNAs is the difficulty targeting the siRNA to the target cell. The signal transduction domain and the MTS of Tat are proposed as targeting agents for siRNA. The DNA sequences disclosed in Example 7 and in SEQ ID NOs. 8, 9 and 10 are exemplary Tat targeting sequences.

The vaccine compositions of the present invention can be administered with additional active agents including, but not limited to, cytokines and adjuvants.

One of skill in the art will recognize that the efficacy, or toxicity, of the vaccine compositions of the present invention, either alone or in combination with other pharmaceuticals, will influence the dose administered to a patient. Those of skill in the art may optimize dosage for maximum benefits with minimal toxicity in a patient without undue experimentation using any suitable method. Additionally, the vaccine compositions of the present invention can be administered in vivo according to any of the methods known to those skilled in the art including, but not limited to, injection, inhalation, infusion and orally or any of the methods described in exemplary texts, such as “Remington's Pharmaceutical Sciences (8^{th} and 15^{th} Editions), the “Physicians' Desk Reference” and the “Merck Index.”

The vaccine compositions can be formulated with any pharmaceutically acceptable excipient as determined to be appropriate by persons skilled in the art. Non-limiting examples of formulations considered with in the scope of the present invention include injectable solutions, lipid emulsions, depots and dry powders. Any suitable carrier can be used in the vaccine composition, which will depend, in part, on the particular means or route of administration, as well as other practical considerations. The pharmaceutically acceptable carriers described herein, for example, vehicles, excipients, adjuvants or diluents, are well known to those who are skilled in the art and are readily available to the public. Accordingly, there are a wide variety of suitable formulations of the vaccine composition of the present invention. The following formulations are exemplary and not intended to suggest that other formulations are not suitable.

Formulations that are injectable are among the preferred formulations. The requirements for effective pharmaceutical carriers for injectable compositions are well known to those of ordinary skill in the art (See Pharmaceutical and Pharmacy Practice, J.B. Lippincott Company, Philadelphia, Pa., Banker & Chalmers, Eds., pp. 238-50, 1982; ASHP Handbook on Injectable Drugs, Toissel, 4^{th} Ed., pp. 622-30, 1986). Such injectable compositions can be administered intravenously or locally, i.e., at or near the site of a disease, or other condition in need of treatment.

Topical formulations are well known to those of skill in the art and are suitable in the context of the present invention. Such formulations are typically applied to skin or other body surfaces.

The vaccine compositions of the present invention, alone or in combination with other suitable components can be made into aerosol formulations to be administered via inhalation. These aerosol formulations can be placed into pressurized acceptable propellants, such as dichlorodifluoromethane, propane, nitrogen and the like. The vaccine compositions of the present invention can also be formulated for dry powder inhalers. They also may be formulated for non-pressured preparations, such as in a nebulizer or an atomizer. Such spray formulations are particularly suitable for spray application to mucosa.

In addition to the above-described pharmaceutical compositions, the vaccine compositions of the present invention can be formulated as inclusion complexes, such as cyclodextrin inclusion complexes, or in liposomes (including modified liposomes such as pegylated and/or targeted liposomes).

It is within the scope of the present invention to provide vaccine compositions to a patient in need thereof through a plurality of routes of administrations using a plurality of formulations.

Additionally, the vaccine compositions of the present invention can be administered to patients in need of induction of specific immune responses according to dosing schedules known to persons skilled in the art, such as physicians. The scope of the present invention is considered to include administration of the vaccine compositions of the present invention either before, concurrent or after the patient has been exposed to an infectious disease organism or evidence of cancer is present. The vaccine compositions of the present invention may be administered in a single dose or as repeated doses.

### EXAMPLES

### Example 1

### Derivatization of Tat to Promote Stimulatory Activities

Conventional immunosuppressive HIV Tat is chemically or physically derivatized to form immunostimulatory Tat*. These Tat proteins are derivatized to reduce or eliminate their immunosuppressive activity, which is verified using the in vitro macrophage bioassay described in Example 6. The chemical and physical methods used to derivatize Tat include, but are not limited to, chemical oxidation and irradiation.

In one embodiment of the present invention, Tat proteins are chemically oxidized using 3% hydrogen peroxide for one hour at approximately 25° C. Other methods for chemical oxidation include: 1 mM to 1 M sodium periodate for one hour at approximately 25° C., 1 mM to 1 M peroxyacids for one hour at approximately 25° C.; 1 mM to 1 M m-chloroperbenzoic acid for one hour at approximately 25° C.; and other chemical and physical oxidative processes known to those skilled in the art. Residual oxidants can be eliminated from the Tat* preparation by adding a suitable biocompatible oxidizable substrate to the Tat* preparation until oxidation is complete. Samples of suitable biocompatible oxidative substances include, but are not limited to, glycerol, carbohydrates and similar compounds known to those in the art.

### Example 2

### Effects of Tat on the Dendritic Cell Lineage

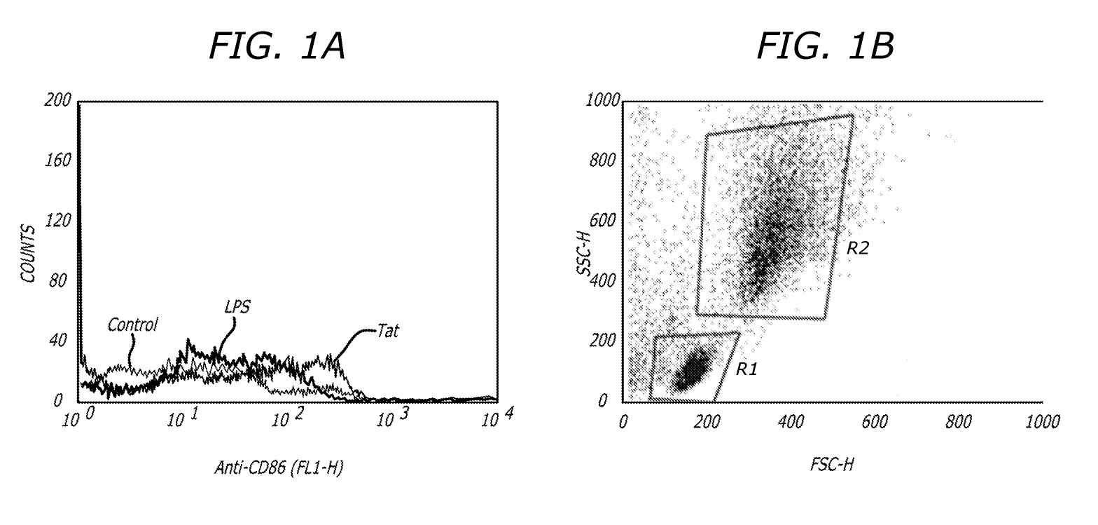

An additional embodiment of the present invention is that Tat induces monocytes committed to the dendritic cell (DC) lineage to enlarge into activated, CD86+ DC APCs (FIG. 1). Human monocytes enriched from PBMCs by Percoll density gradient separation and adherance to anti-CD14 coated magnetic beads (Dynabeads M-450, Dynal Biotech) were committed to differentiate into DCs through five days of culture in GM-CSF (100 ng/mL) and IL-4 (100 ng/mL). Committed DCs were cultured overnight either in medium alone (Control), LPS (100 ng/mL), or Tat (50 nM), after which they were stained with an anti-CD86 antibody (BD Pharmingen) and analyzed by FACScan for CD86 induction (left panel) or generalized activation (right panel, enlargement into box R2, shown for Tat-stimulated cells). The MFIs for CD86 expression are 9 (Control), 30 (LPS), and 187 (Tat), CD86 being a specific determinant of DC activation.

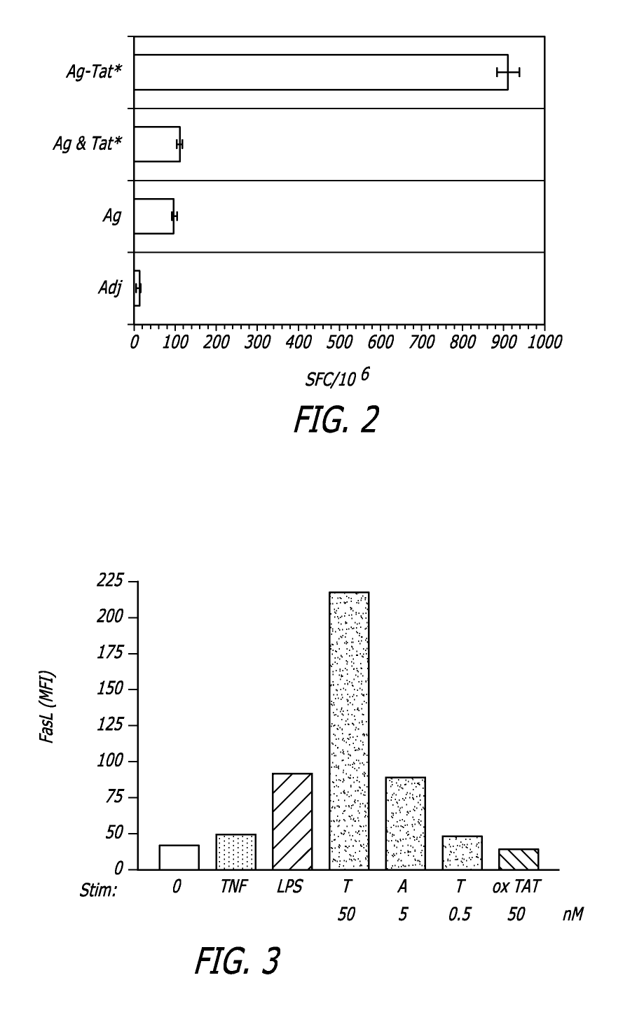

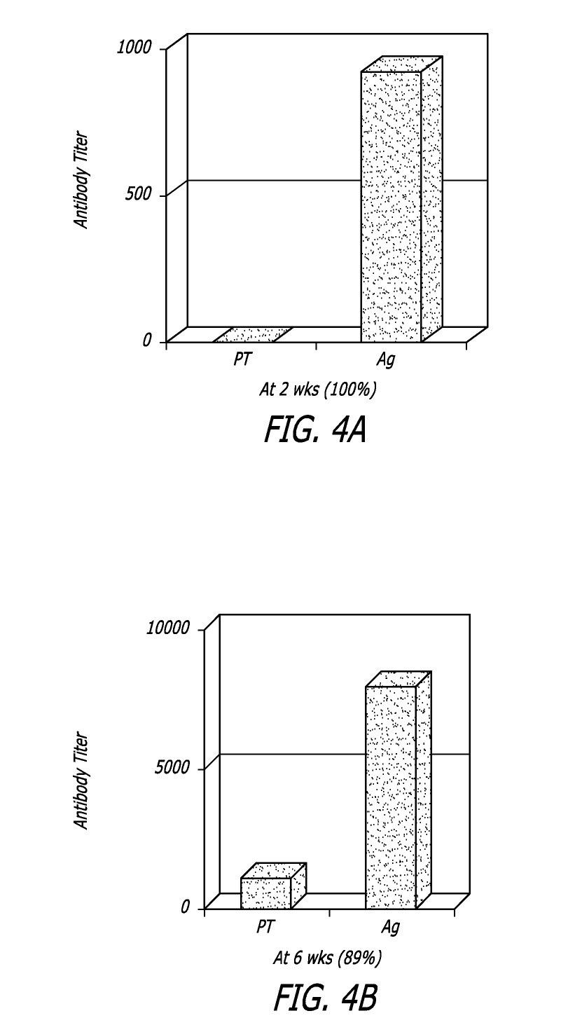

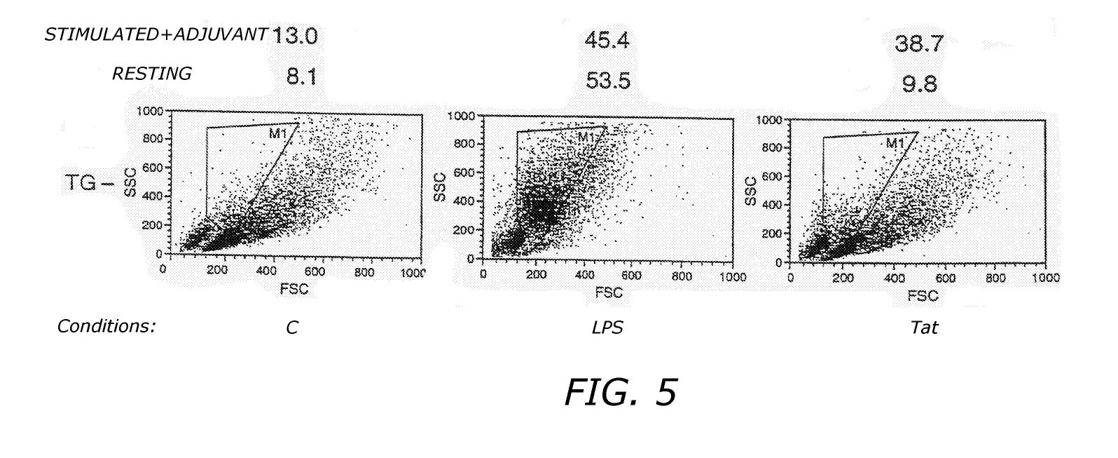

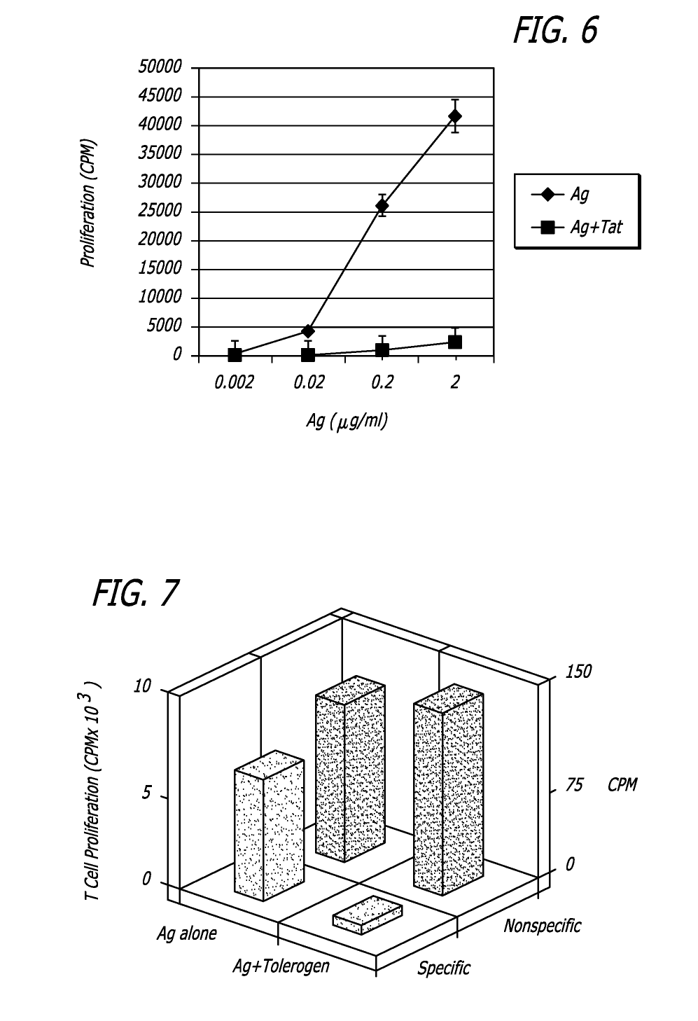

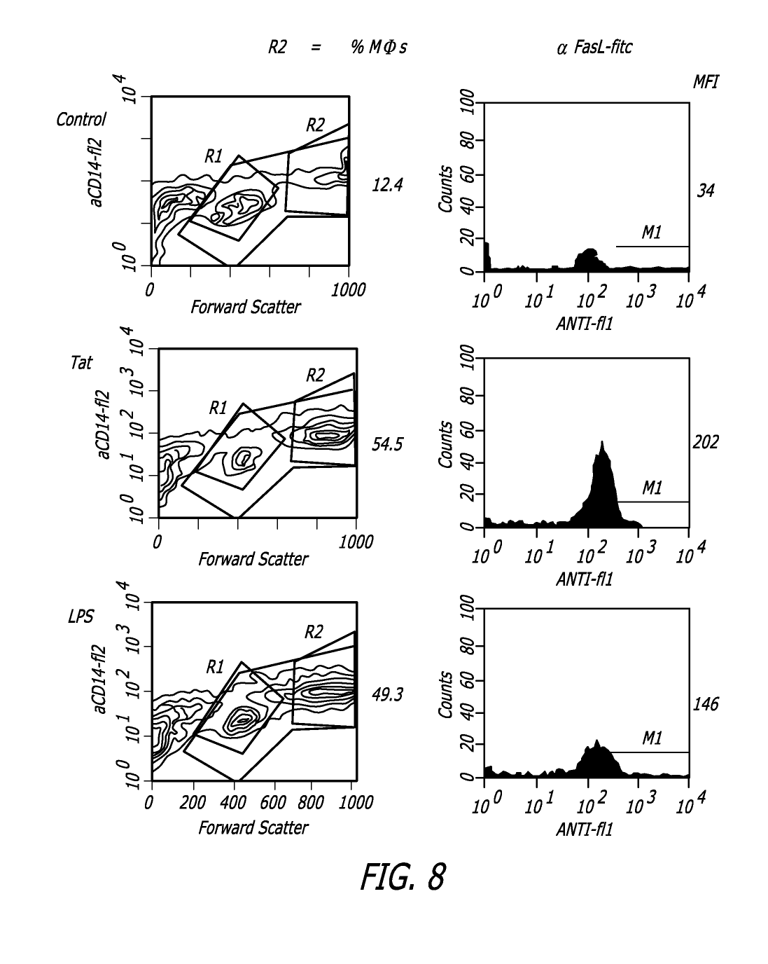

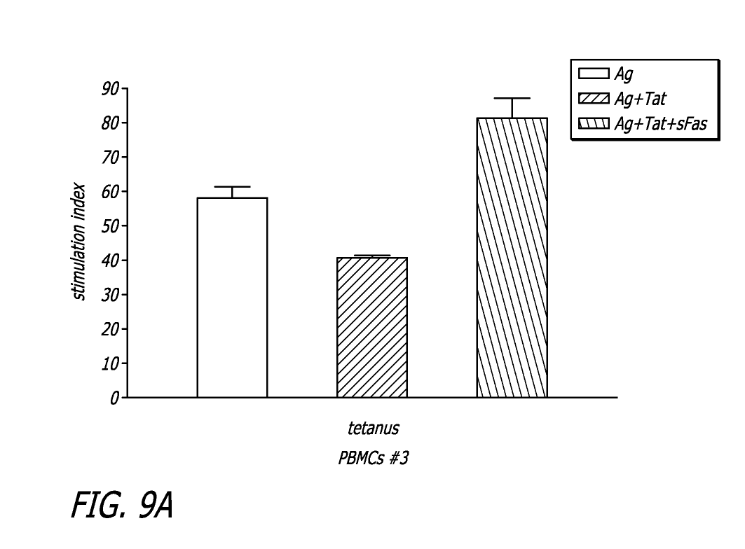

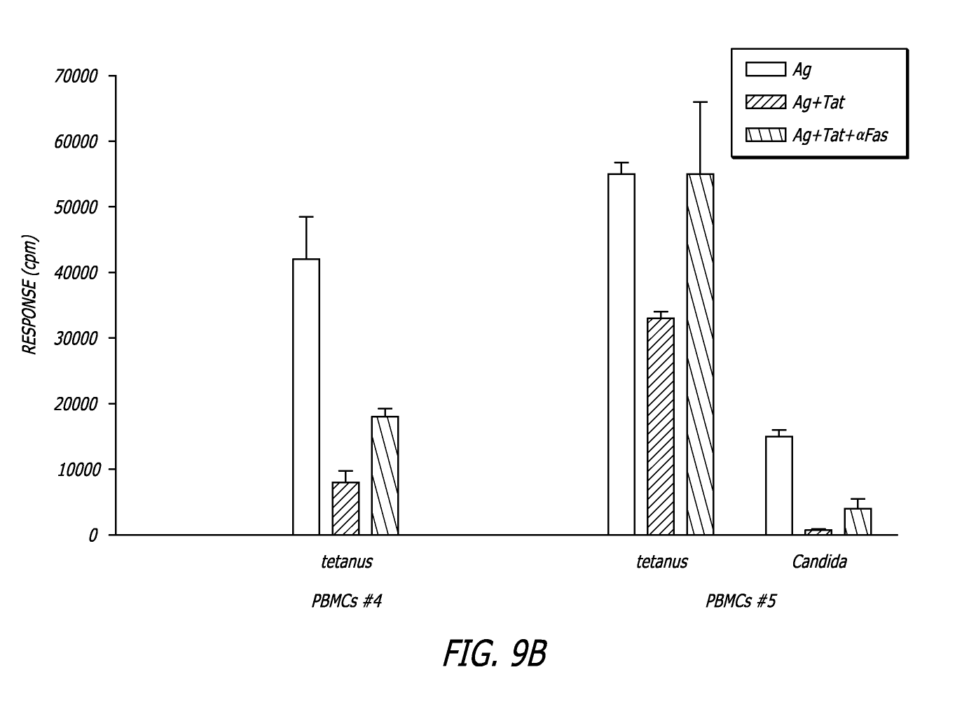

Derivitzed Tat reduces AReg differentiation and potently enhances antigen-specific activation of CTLs (FIG. 2). Tat is chemically derivatized by oxidation (Tat* or ox-Tat) so that it does not induce ARegs from monocyte APC precursors (FIG. 3). Ten micrograms of Tat/p24 Tat*-Ag conjugate (Ag-Tat*) was administered into the flanks of Balb/C mice in adjuvant on day 0 and day 7. Experimental groups were comparatively immunized in adjuvant with 5 μg of p24 in one flank and 5 μg derivatized Tat in the other flank (Ag & Tat*), or 10 μg of p24 in adjuvant (Ag). Control mice were given two injections of adjuvant. Four mice were treated in each group. At day 14, draining lymph node cells from each animal were harvested and re-stimulated overnight in cultures of irradiated Ap24 (H-2d cells stably transfected to express antigen p24) cells or control non-transfected cells. Cytotoxic T lymphocyte activity was quantitated as the number of γ-interferon secreting spot forming colonies (SFC)/10⁶ plated cells using ELISPOT assays. The background with non-transfected re-stimulators, which was in all cases <10 SFC/10⁶, is subtracted from each point. The results are indicative of three similar experiments.

### Example 3

### Re-Activation of Suppressed T Lymphocytes by Vaccine Compositions

As depicted in FIG. 15, alloreactive human peripheral blood mononuclear cells (PBMC) were maintained in interleukin-2 rich medium (10 μg/mL) for 2 weeks. A that time, the cells were harvested and re-stimulated in the presence of 10³ fresh irradiated allogeneic PBMCs (Ag) either in the absence (APC) or presence (APC+PINS) of the vaccine composition of the present invention (100 ng/mL). Additional cytokine stimuli were added as indicated at 10 μg/mL each. GM stands for granulocyte macrophage colony stimulating factor (GM-CSF).

Cytokines alone were unable to stimulate proliferation of the alloreactive PBMCs however addition of the vaccine composition (PINS) led to induction of significant T cell proliferation (FIG. 15).

### Example 4

### Efficacy of an E7 Vaccine Composition Against Cervical Carcinoma in Mice

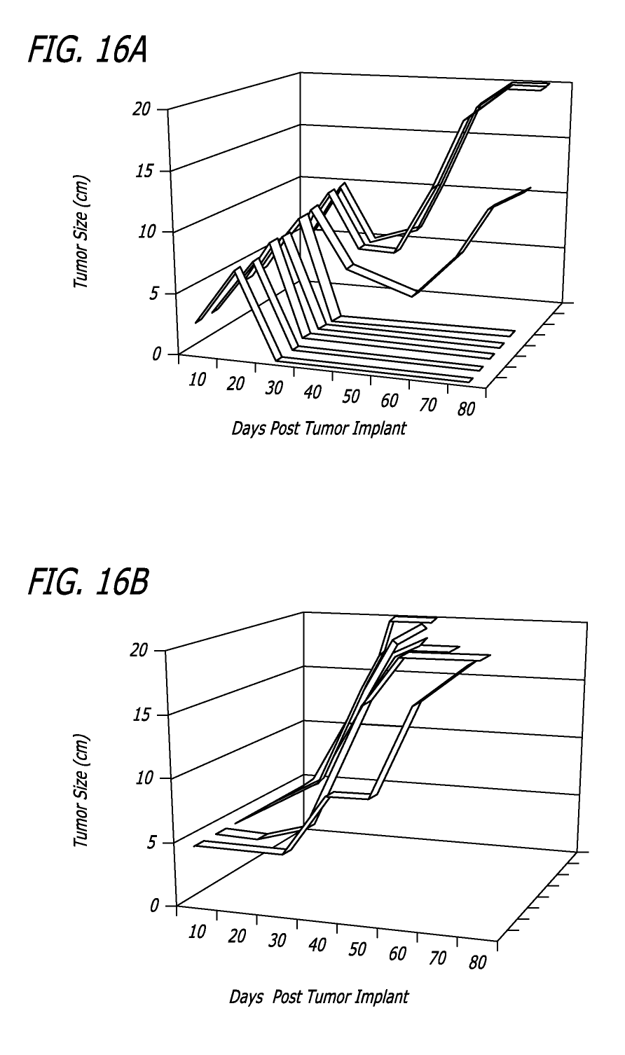

Mice (C57BL/6) in groups of eight, were implanted with 1×10⁶ syngeneic E7-transformed epithelial cells subcutaneously. Two days and five days following implantation, each mouse was immunized in the flank with 1 μg of either cancer vaccine composition E7PINS (FIG. 16A) or E7 antigen alone (FIG. 16B) in phosphate buffered saline. The E7PINS cancer vaccine composition was constructed in a pCMV-DsRed-Express vector (Catalog #632416, BD Biosciences, Palo Alto, Calif.) containing a promoter, the E7 nucleotide sequence, modified Tat* (exons 1 and 2) and a poly adenine sequence. Modified Tat* was made by site-directed mutagenesis of native Tat by changing the proline residues at positions 6, 10 and 14 to glycine residues. Every 10 days the tumor size (cm) was measured and recorded. At 80 days, the surviving mice were euthanized according to standard guidelines.

Immunization with E7 alone in mice bearing cervical carcinoma tumors resulted in survival of only 25% of the mice at 70 days (FIG. 16B), however immunization with the E7PINS vaccine composition led to survival of 100% of the animals at day 70 (FIG. 16A).

### Example 5

### Sequence and Homology Features of the Tat Protein

The complete amino acid sequence of HIV-1 Tat encoded by exons 1 and 2 of the Tat gene is listed below:

```
ATG GAG CCC GTG GAC CCT CGC CTG GAG CCC TGG AAG CAC CCG GGC AGC
Met Glu Pro Val Asp Pro Arg Leu Glu Pro Trp Lys His Pro Gly Ser
1 5 10/30 15
CAG CCC AAG ACC GCC TGC ACC ACA TGT TACT GC AAG AAG TGC TGC TTC
Gln Pro Lys Thr Ala Cys Thr Thr Cys Tyr Cys Lys Lys Cys Cys Phe
                        20/60 25 30/90
CAC TGC CAG GTG TGC TTC ACC AAG AAG GCC TTG GGC ATC AGC TAC GGC
His Cys Gln Val Cys Phe Thr Lys Lys Ala Leu Gly Ile Ser Tyr Gly
                35 40/120 45
CGC AAG AAG CGC CGG CAG CGC CGC CGG FCC CCT GAG GAC AGC CAG ACC
Arg Lys Lys Arg Arg Gln Arg Arg Arg Ala Pro Glu Asp Ser Gln Thr
        50/150 55 60/180
CAC CAG GTG AGC CCT CCC AAG CAG CCC GCT CCA CAG TTC CGC GGC GAC
His Gln Val Ser Pro Pro Lys Gln Pro Ala Pro Gln Phe Arg Gly Asp
65 70/210 75 80/240
CCT ACC GGT CCC AAG GAG AGC AAG AAG AAG GTG GAG CGC GAG ACC GAG
Pro Thr Gly Pro Lys Glu Ser Lys Lys Lys Val Glu Arg Glu Thr Glu
                                85 90/270 95
ACC CAT CCC GTC GAC SEQ ID NO. 1
Thr His Pro Val Asp SEQ ID NO. 2
                        100/300
```

The Tat of the present invention has a proline (P) rich segment near the amino terminus (amino acids 3-19):

(SEQ ID NO. 3)
Pro Val Asp Pro Arg Leu Glu Pro Trp Lys His Pro Gly Ser Gln Pro Lys

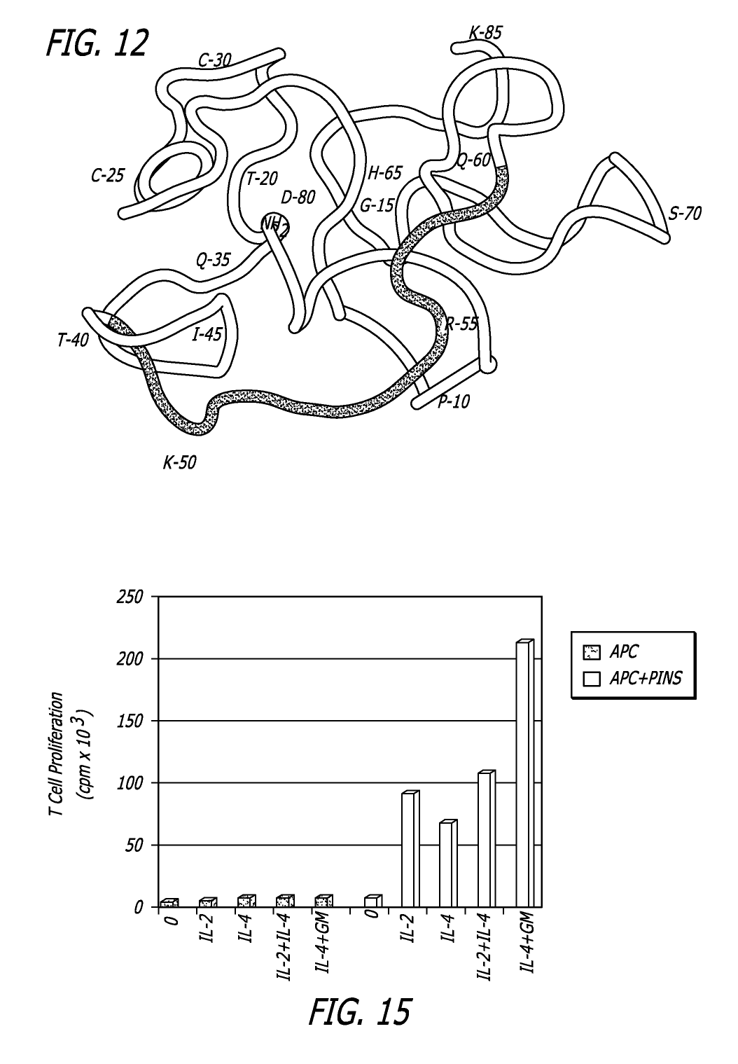

This highly conserved region of HIV-1 Tat is a canonical SH3 binding domain (FIG. 12).

The mouse hairless (hr) gene also has an SH3 binding motif of amino acids 176-196:

(SEQ ID NO. 4)
Pro Cys Asp Trp Pro Leu Thr Pro Asp Pro Trp Val Tyr Ser Gly Ser Gln
Pro Lys Val Pro

Homology exists between the human Tat SH3 binding domain (SEQ ID NO. 3) and the SH3 binding domain of the mouse hr gene (SEQ ID NO. 4):

| | |
|---|---|
| Human | 3 Pro Val Asp Pro Arg Leu Glu Pro Trp Lys His Pro 14 |
| Mouse | 180 Pro Leu Thr Pro Asn---------Pro Trp Val Tyr Ser 189 |
| Human | 15 Gly Ser Gln Pro 18 |
| Mouse | 190 Gly Ser Gln Pro 193 |

Variants of Tat found in simian lentiviruses that do not cause immunodeficiency do not have an SH3 binding domain but instead have the following proline-flanked sequence:

(SEQ ID NO. 5)
Pro Leu Arg Glu Gln Glu Asn Ser Leu Glu Ser Ser Asn Glu Arg Ser Ser
Cys Ile Leu Glu Ala Asp Ala Thr Thr Pro

The human equivalent of the simian sequence above (SEQ ID NO. 5) is:

(SEQ ID NO. 6)
Ser Asn Glu Arg Ser Ser Cys Glu Leu Glu Val

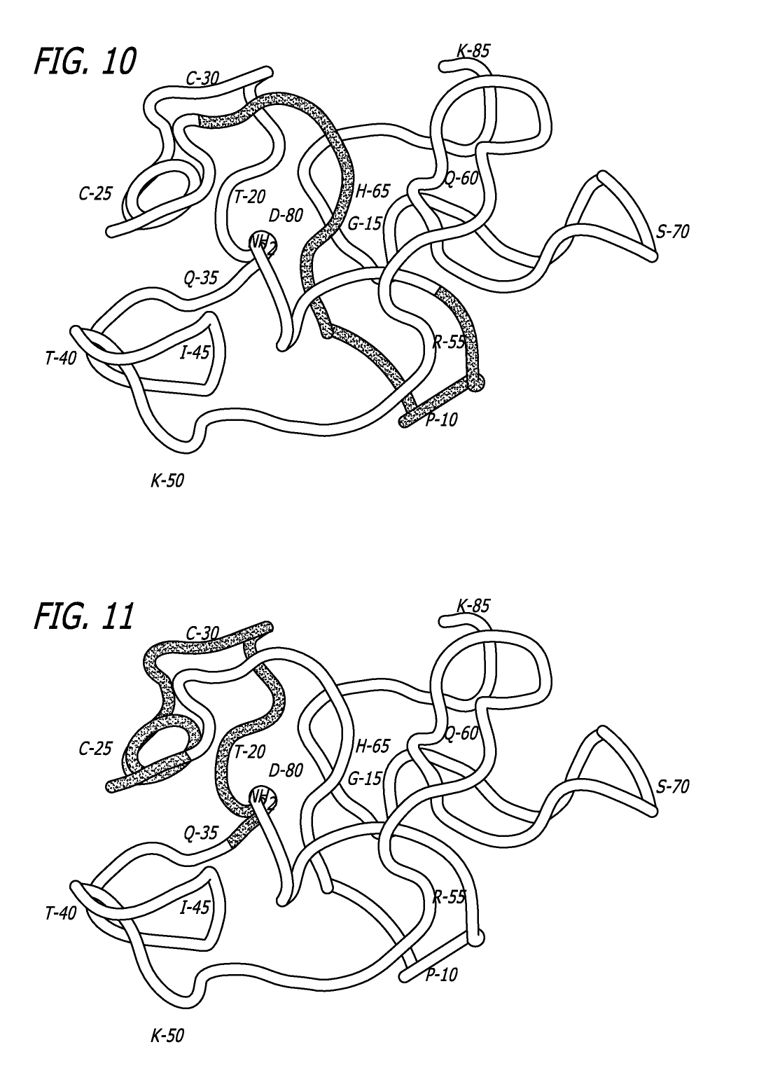

Another region of interest is a cysteine-rich proposed ligand binding domain (amino acids 22-37) which contains seven cysteines (FIG. 10).

(SEQ ID No. 7)
Cys Thr Thr Cys Tyr Cys Lys Lys Cys Cys Phe His Cys Gln Val Cys

Additionally, it is known that Tat contains a membrane translocation domain (MTS) (FIG. 11).

A modified Tat* comprising the insertion of the immunostimulatory human equivalent of simian lentivirus Tat (SEQ ID NO. 6) into native immunosuppressive Tat.

```
SEQ ID NO. 11
Met Glu Pro Ser Asn Glu Arg Ser Ser Cys Glu Leu Glu Val Pro Lys
1 5 10 15
Thr Ala Cys Thr Thr Cys Tyr Cys Lys Lys Cys Cys Phe His Cys Gln
                                20 25 30
Val Cys Phe Thr Lys Lys Ala Leu Gly Ile Ser Tyr Gly Arg Lys Lys
                                35 40 45
Arg Arg Gln Arg Arg Arg Ala Pro Glu Asp Ser Gln Thr His Gln Val
                                50 55 60
Ser Pro Pro Lys Gln Pro Ala Pro Gln Phe Arg Gly Asp Pro Thr Gly
                              s 65 70 75
Pro Lys Glu Ser Lys Lys Lys Val Glu Arg Glu Thr Glu Thr His Pro
                                80 85 90
Val Asp
        94
```

### Example 6

### In Vitro Bioassay for Monocyte Differentiation

The in vitro ultra-sensitive monocyte Tat bioassay of the present invention is used to assess the immunosuppressant or immunostimulatory activity of the Tat proteins used in vaccine compositions of the present invention. This assay utilizes fresh monocyte cells substantially purified from human peripheral blood using standard density gradient enrichment procedures or other cell isolation protocols known in the art. The substantially purified monocytes are washed and then cultured in RPMI-1640 supplemented with 10% FBS at 37° C.

The in vitro ultra-sensitive monocyte Tat bioassay is performed using a positive control (FasL, inducing compound) and a negative control (no active compound is added to the culture). Suitable positive controls include, but are not limited to, lipopolysaccharide (LPS) and or tissue necrosing factor (TNF-α) at a final concentration of 100 ng/mL and 50 ng/mL, respectively. Test samples (Tat preparations) are run at final concentrations from 50 pM to 50 nM and include Tat, ox-Tat, NICE and other Tat derivatives and mutants.

The test samples and controls are individually mixed with the substantially pure monocytes seeded at a density of 10⁶ cells/mL in round bottom tubes containing RPMI-1640 with 10% FBS (herein referred to collectively as assay cultures). The assay cultures are then incubated for a suitable period of time, preferably from five to six days, at 37+ C., in a 5% CO₂ environment.

At the end of the incubation period, cells are removed from each assay culture and the presence of any induced FasL expression (for measurement of differentiation into ARegs) or CD86 expression (for differentiation in dendritic cells) is detected by staining with an anti-FasL or anti-CD86 antibodies and appropriate fluorescent detection agents. After the substantially pure macrophages have been stained, the fluorescence is detected using a fluorescence activated cell sorter (FACS) system. Control staining is performed using the fluorescent detection system alone and subtracted from the specific anti-FasL or anti-CD86 staining seen in the assay cultures. The greater the percentage of FasL positive cells in a given assay culture, the more immunosuppressant the test sample in the assay culture is. Conversely, if the assay culture contains a predominance of CD86 positive cells, the test sample is identified to be immunostimulatory. Negative controls should always remain non-reactive with the antibodies and the positive control should fall within predetermined ranges.

### Example 7

### siRNA Targeting Domains

HIV-1 Tat SH3 targeting domain:

(SEQ ID NO. 8)
ccagtagatc ctagactaga gccctggaag catccaggaa
gtcagcctaa

Mouse strain hairless SH3 targeting domain:

(SEQ ID NO. 9)
ccatgtgact ggcccctgac cccgcacccc tgggtatact ccgggggcca gcccaaagtg
ccc

Targeting domain from the human equivalent of the simian non-immunosuppressive Tat:

(SEQ ID NO. 10)
agcaacgagc ggagttcctg cgagttagag gtg

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

I claim:

1. A Tat-based vaccine composition comprising at least one antigen coupled to at least one immunostimulatory lentivirus trans-activator of transcription (Tat) molecule.

2. The Tat-based vaccine composition of claim 1 wherein said antigen comprises a full length protein or a fragment thereof.

3. The Tat-based vaccine composition of claim 1 wherein said antigen is a cancer antigen or an infectious disease antigen.

4. The Tat-based vaccine composition of claim 3 wherein said cancer antigen is an antigen associated with cell growth.

5. The Tat-based vaccine composition of claim 3 wherein said cancer antigen is human papilloma virus E7.

6. The Tat-based vaccine composition of claim 1 wherein said immunostimulatory lentivirus Tat is oxidized human immunodeficiency virus-1 (HIV-1) Tat.

7. The Tat-based vaccine composition of claim 1 wherein said immunostimulatory lentivirus Tat is the HIV-1 Tat wherein the amino acid proline at positions 6, 10 and 14 of SEQ ID NO. 1 is replaced with the amino acid glycine.

8. The Tat-based vaccine composition of claim 1 wherein said immunostimulatory lentivirus Tat comprises the amino acid sequence of SEQ ID NO. 11.

9. The Tat-based vaccine composition of claim 1 wherein said immunostimulatory lentivirus Tat and said antigen are linked through genetic engineering of their DNA to provide a recombinant protein.

10. A method for treating disease comprising administering at least one Tat-based vaccine composition to a patient in need thereof.

11. The method for treating disease according to claim 10 wherein said administering at least one Tat-based vaccine composition to a patient in need thereof further comprises administering said at least one Tat-based vaccine composition for the treatment of cancer.

12. The method for treating disease according to claim 10 wherein said administering at least one Tat-based vaccine composition to a patient in need thereof further comprises administering said at least one Tat-based vaccine composition for the treatment of infectious disease.

13. A Tat-based vaccine composition comprising immunostimulatory lentivirus Tat.

14. The Tat-based vaccine composition of claim 13 further comprising at least one antigen wherein said antigen is a full length protein or a fragment thereof.

15. The Tat-based vaccine composition of claim 13 wherein said immunostimulatory lentivirus Tat is oxidized HIV-1 Tat.

16. The Tat-based vaccine composition of claim 13 wherein said immunostimulatory lentivirus Tat is HIV-1 Tat wherein the amino acid proline at positions 6, 10 and 14 of SEQ ID NO. 1 is replaced with the amino acid glycine.

17. The Tat-based vaccine composition of claim 13 wherein said immunostimulatory lentivirus Tat comprises the amino acid sequence of SEQ ID NO. 11.


SEQUENCE LISTING (11 sequences)

SEQ ID NO: 1 - protein, 101 aa, Human immunodeficiency virus type 1
MEPVDPRLEPWKHPGSQPKTACTTCYCKKCCFHCQVCFTKKALGISYGRKKRRQRRRAPEDSQTHQVSPPKQPAPQFRGDPTGPKESKKKVERETETHPVD

SEQ ID NO: 2 - DNA, 303 bp, Human immunodeficiency virus type 1
atggagcccgtggaccctcgcctggagccctggaagcacccgggcagccagcccaagaccgcctgcaccacatgttactgcaagaagtgctgcttccactgccaggtgtgcttcaccaagaaggccttgggcatcagctacggccgcaagaagcgccggcagcgccgccgggcccctgaggacagccagacccaccaggtgagccctcccaagcagcccgctccacagttccgcggcgaccctaccggtcccaaggagagcaagaagaaggtggagcgcgagaccgagacccatcccgtcgac

SEQ ID NO: 3 - protein, 17 aa, Human immunodeficiency virus type 1
PVDPRLEPWKHPGSQPK

SEQ ID NO: 4 - protein, 21 aa, Human immunodeficiency virus type 1
PCDWPLTPDPWVYSGSQPKVP

SEQ ID NO: 5 - protein, 27 aa, Simian immunodeficiency virus
PLREQENSLESSNERSSCILEADATTP

SEQ ID NO: 6 - protein, 11 aa, Human immunodeficiency virus type 1
SNERSSCELEV

SEQ ID NO: 7 - protein, 16 aa, Human immunodeficiency virus type 1
CTTCYCKKCCFHCQVC

SEQ ID NO: 8 - DNA, 50 bp, Human immunodeficiency virus type 1
ccagtagatcctagactagagccctggaagcatccaggaagtcagcctaa

SEQ ID NO: 9 - DNA, 63 bp, Mus musculus
ccatgtgactggcccctgaccccgcacccctgggtatactccgggggccagcccaaagtgccc

SEQ ID NO: 10 - DNA, 33 bp, Simian immunodeficiency virus
agcaacgagcggagttcctgcgagttagaggtg

SEQ ID NO: 11 - protein, 98 aa, Artificial
  Modified immunostimulatory Tat
MEPSNERSSCELEVPKTACTTCYCKKCCFHCQVCFTKKALGISYGRKKRRQRRRAPEDSQTHQVSPPKQPAPQFRGDPTGPKESKKKVERETETHPVD